(19) United States
(12) Patent Application Publication
(10) Pub. No.: US 2008/0189046 A1
(43) Pub. Date: Aug. 7, 2008

(54) Optical tool with dynamic electromagnetic radiation and a system and method for determining the position and/or motion of an optical tool

(75) Inventors: Jonas Ove Philip Eliasson, Valby (DK); Niels Agersnap Larsen, Kongens Lyngby (DK); Jens Bastue, Virum (DK); Jens Wagenblast Stubbe Ostergaard, Lejre (DK)

Correspondence Address: PILLSBURY WINTHROP SHAW PITTMAN, LLP, P.O. BOX 10500, MCLEAN, VA, 22102, US

(73) Assignee: O-PEN A/S, Copenhagen (DK)

(21) Appl. No.: 11/701,520

(22) Filed: Feb. 2, 2007

(51) Int. Cl. G01B 11/14; G01C 19/00; G01N 33/48

(52) U.S. Cl. 702/19; 356/614; 702/150; 73/514.01

(57) ABSTRACT

A tool configured to emit electromagnetic radiation therefrom such that one or more aspects of the emission of the electromagnetic radiation may be varied as a function of time. By varying the emission of the electromagnetic radiation as a function of time, the tool may enable information related to its position and/or motion to be determined with an enhanced specificity based on detection of the emitted electromagnetic radiation. For example, the varying emission of the electromagnetic radiation may enable a three dimensional position of the tool to be determined, may enable the position of the tool to be determined in three rotational degrees of freedom, and/or may enable time derivatives of these (and other) position information to be determined to quantify motion of the tool. In some implementations, the emission of the electromagnetic radiation may be varied such that position and/or motion information related to a plurality of tools may be determined simultaneously (or substantially simultaneously).

### FIELD OF THE INVENTION

The invention relates to tools that emit electromagnetic radiation to enable the detection of information related to the position and/or motion of the tools, and to systems and methods for determining information related to the position and/or motion of tools that emit electromagnetic radiation.

### BACKGROUND OF THE INVENTION

The implementation of tools that emit (or otherwise interact with) electromagnetic radiation with detection system capable of determining information related to the position of such tools is known. However, existing systems generally provide a relatively limited amount of information regarding the position of an emitting tool. For example, a system may only determine a location at which the tool is pointed. As another example, a system may only determine a location of a tool if the tool is at or near an interface surface associated with the tool. Generally, the detection of an accurate three-dimensional position of a tool is not enabled by conventional systems. Further, conventional systems may not enable a robust detection of the position of a tool in three rotational degrees of freedom. Other drawbacks with existing systems exist.

### SUMMARY

One aspect of the invention may relate to a tool configured to emit electromagnetic radiation therefrom such that one or more aspects of the emission of the electromagnetic radiation may be varied as a function of time. By varying the emission of the electromagnetic radiation as a function of time, the tool may enable information related to its position and/or motion to be determined with an enhanced specificity based on detection of the emitted electromagnetic radiation. For example, the varying emission of the electromagnetic radiation may enable a three dimensional position of the tool to be determined, may enable the position of the tool to be determined in three rotational degrees of freedom, and/or may enable time derivatives of these (and other) position information to be determined to quantify motion of the tool. In some implementations, the emission of the electromagnetic radiation may be varied such that position and/or motion information related to a plurality of tools may be determined simultaneously (or substantially simultaneously). The information related to the position and/or motion of the tool may be implemented as input to an electronic system (e.g., a gaming system, an information management system, an electronic control system, etc.).

In some implementations, varying one or more aspects of the emission of electromagnetic radiation may include varying one or more of the directionality, the amplitude, the frequency, amplitude modulation, frequency modulation and/or other aspects of the emission of electromagnetic radiation. In some instances, one or more aspects of the emission of electromagnetic radiation may be varied as a function of time in a predetermined manner. In addition to this dynamic electromagnetic radiation emitted by the tool, the tool may further emit a beam of electromagnetic radiation as a “pointer beam.” The pointer beam may provide a user interacting with the tool with a reference of where the tool is currently being pointed.

In some implementations, the tool may emit electromagnetic radiation in a dynamic spatial pattern that changes as time passes. This may include expanding and/or contracting the pattern as time passes. In some such implementations, the tool may emit one or more beams of electromagnetic radiation that are scanned in a spiral pattern that expands and/or contracts as time passes. The spiral pattern may include a circular spiral pattern, a square spiral pattern, a triangular spiral pattern, and/or other spiral patterns. As another possibility, the tool may emit electromagnetic radiation in a predetermined shape that expands and/or contracts as time passes. For instance, the tool may emit a circle, a square, a triangle, a cross, and/or other shapes that expand and/or contract as time passes. In some instances, the emission of electromagnetic radiation may be pulsed (e.g., amplitude modulated) to emit bursts of electromagnetic radiation. As is discussed further below, the pulse rate (e.g., the frequency of the amplitude modulation) may be constant, or may be varied.

In some implementations, an input system configured to determine information related to the position and/or motion of the tool may include the tool, a detection arrangement, a processor, and/or other components. A user may interact with the tool (e.g., hold the tool and position and/or move the tool in relation to other components of the system, etc.) to input information to the input system. The detection arrangement may receive at least a portion of the electromagnetic radiation emitted by the tool, and may generate one or more output signals based on one or more properties of the received electromagnetic radiation. For example, the one or more output signals may be related to the positions in an interface surface associated with the detection arrangement that receive the electromagnetic radiation emitted by the tool. The processor may receive the one or more output signals generated by the detection arrangement, and based at least in part on the one or more output signals, may determine information related to the position of the tool.

For example, the processor may determine a center of the spatial distribution of the zones on the interface surface that receive electromagnetic radiation from the tool at a given point in time. This location may coincide with, or be otherwise related to, a point on the interface surface at which the user was pointing the tool at the given point in time. Further, one or more aspects of the shape and size of the zones on the interface surface that receive electromagnetic radiation from the tool may be a function of the position of the tool with respect to the interface surface at the given point in time. For example, unless the tool pointed along an axis that is perpendicular to the interface surface, the zones may not represent a cross-section of the pattern of emitted electromagnetic radiation. Instead, the zones may be elongated (e.g., from a circular cross-section to an elliptical zone of illumination on the interface surface) in a direction that corresponds to direction of an angle between the axis along which the tool is being pointed and an axis that is perpendicular to interface surface (e.g., this angle may account for the pitch and yaw of the tool with respect to the axis perpendicular to the interface surface). The amount of elongation of the zones may correspond to the magnitude of the angle (e.g., the larger the angle, the more elongated the zones may become). Accordingly, based on the deformation of the pattern of electromagnetic radiation, the direction from which the electromagnetic radiation has emanated (e.g., tool) may be determined.

As was mentioned above, in some instances, the spatial distribution of the emission of electromagnetic radiation by the tool may be varied over time (e.g., to expand and/or contract over time). By analyzing the change in size of the pattern of electromagnetic radiation formed by the zones of the interface surface that receive electromagnetic radiation over time (e.g., from a first given point in time to a second given point in time), the distance from the interface surface to the tool may be determined. More specifically, if the field of emission of the electromagnetic radiation emitted by the tool is varied as a function of time in a predetermined manner (e.g., at a predetermined rate), that corresponding changes in the size of the pattern of electromagnetic radiation formed by the zones of the interface surface receiving the electromagnetic radiation will increase as the tool is moved away from the interface surface. Similarly, as the tool is moved toward interface surface, the rate of change in size of the patterns of electromagnetic radiation on the interface surface will decrease. Provided that the function being implemented by the tool to vary the size of the field of emission (e.g., the rate of expansion and/or contraction) is known, the relationship between the variance of the spatial distribution of the emitted electromagnetic radiation and the variance of the size of the corresponding patterns of electromagnetic radiation formed on the interface surface may be leveraged to determine the distance between the tool and interface surface (e.g., by triangulation).

Upon determination of the distance of the tool from the interface surface, the position of the tool in three dimensions may be determined (e.g., the determined distance along the determined optical axis from the center of the illuminated zone on the interface surface). Further, the orientation of the tool in two degrees of freedom may be determined (based on the orientation of the axis along which the tool is being pointed). This determination may be referred to as the “tilt” of the tool with respect to the interface surface.

In some embodiments, the rotational orientation of the tool about the axis along which the tool is being pointed (e.g., also referred to as the “roll” of tool 12) may further be determined. To enable this determination, the field of emission of the tool may be marked in some way. For example, an irregularity may be provided at one location on the boundary of the field (e.g., a protrusion, an intrusion, etc.), or within the field (e.g., a “hole), that may be identified in the corresponding zone created on the interface surface. As another example, the electromagnetic radiation emitted by the tool may be filtered in such a way as to mark the electromagnetic radiation. For instance, electromagnetic radiation in one area of the field of emission may be provided with a different frequency, intensity, and/or modulation than other areas of the field. Other mechanisms for marking the electromagnetic radiation emitted by tool may be employed.

Based on the orientation of the marked area in the zone formed on or near the interface surface by the electromagnetic radiation emitted by the tool, roll of the tool may be determined. This determination, in conjunction with the other determinations, discussed above, related to the position of the tool with respect to the interface surface may enable the determination of the position of the tool in six degrees of freedom (e.g., three translational degrees of freedom and three rotational degrees of freedom). Some or all of this positional information may be used to input information to the input system that includes the tool and the detection arrangement.

The determined information related to the position of the tool may further be implemented to determine information related to the motion of tool as the user interacts with it. For example, determinations of position may be aggregated to determine time derivatives of the position of the tool such as velocity, acceleration, jerk, etc. These time derivatives may be determined for translational and/or rotational movement of the tool. Such aggregations of position information may be achieved using conventional mechanisms for determining time derivatives of position. These values (velocity, acceleration, jerk, etc.) may also be used as a mechanism for enabling the user to input information to the input system.

### BRIEF DESCRIPTION OF THE DRAWINGS

FIG. 1 illustrates an input system, in accordance with one or more embodiments of the invention.

FIG. 2 illustrates a detection arrangement, according to one or more embodiments of the invention.

FIG. 3 illustrates a detection arrangement, in accordance with one or more embodiments of the invention.

FIG. 4 illustrates a detection arrangement, according to one or more embodiments of the invention.

FIG. 5 illustrates an input system, in accordance with one or more embodiments of the invention.

### DETAILED DESCRIPTION

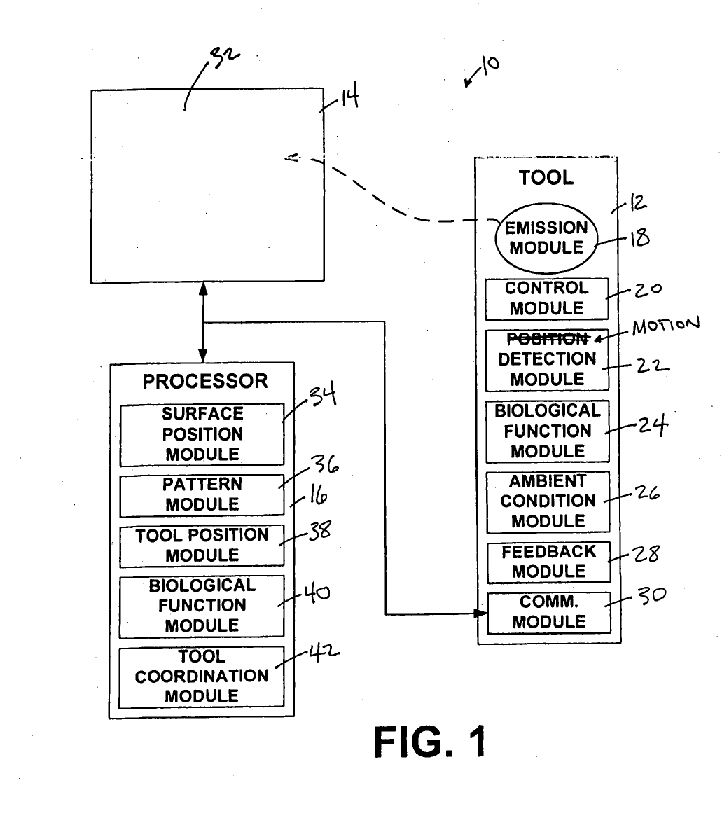

FIG. 1 illustrates an input system 10 that enables a user to input information to an electronic system in communication with input system 10, in accordance with one or more embodiments of the invention. System 10 may include a tool 12, a detection arrangement 14, a processor 16, and/or other components. The user may interact with tool 12 (e.g., hold tool 12 and position and/or move tool 12 in relation to other components of system 10) to input information to input system 10. In some implementations, tool 12 may emit electromagnetic radiation therefrom. Detection arrangement 14 may receive at least a portion of the electromagnetic radiation emitted by tool 12, and may generate one or more output signals based on one or more properties of the received electromagnetic radiation. Processor 16 may receive the one or more output signals generated by detection arrangement 14, and based at least in part on the one or more output signals may determine information related to the position of tool 12. The information determined by processor 16 may include a location at which the user is pointing tool 12, a three dimensional position of tool 12, a position of tool 12 in three rotational degrees of freedom, and/or other information related to the position of tool 12. In some instances, system 10 may enable information related to the position of multiple tools similar to tool 12 to be determined simultaneously (or substantially simultaneously).

In some embodiments of the invention, tool 12 may include an emission module 18, a control module 20, a motion detection module 22, a biological function module 24, an ambient conditions module 26, a feedback module 28, a communication module 30, and/or other modules and/or components. Although tool 12 is illustrated in FIG. 1 as a single, integrated device, this is not intended to be limiting. In some implementations, one or more of modules 18, 20, 22, 24, 26, 28, and/or 30 may be provided separately from each other (e.g., in separate, non-integrated devices), and a communication link may be formed between the separated modules to enable modules 18, 20, 22, 24, 26, 28, and/or 30. This communication link may be formed via a hard-wired connection, and/or through a wireless connection (e.g., implementing WiFi, WiMax, Bluetooth, etc.). As is discussed further below, various ones of modules 18, 20, 22, 24, 26, 28, and/or 30 may provide functionality to system 10 that enhances the ultimate determination of information related to the position of tool 12 and/or enables the determination of other information related to tool 12, a user interacting with tool 12, ambient conditions surrounding tool 12, and/or other information. It should be appreciated that the implementations of tool 12 shown in FIG. 1 including all of modules 18, 20, 22, 24, 26, 28, and/or 30, and their accompanying functionalities, are provided for illustrative purposes only. In some implementations, tool 12 may not include all of modules 18, 20, 22, 24, 26, 28, and/or 30.

Emission module 18 may be configured to emit electromagnetic radiation such that one or more aspects of the emission of electromagnetic radiation can be varied. For example, emission module 18 may vary one or more of the directionality, the spatial distribution, the amplitude, the frequency, amplitude modulation, frequency modulation and/or other aspects of the emission of electromagnetic radiation. In some instances, one or more aspects of the emission of electromagnetic radiation may be varied as a function of time in a predetermined manner. In addition to this dynamic emission of electromagnetic radiation by emission module 18, emission module 18 may further emit a beam of electromagnetic radiation as a “pointer beam.” The pointer beam may be emitted in a constant (or substantially constant) direction. This direction may be referred to as the direction in which the user is pointing tool 12.

Emission module 18 may include one or more sources configured to generate the electromagnetic radiation emitted by emission module 18 and/or one or more optical elements configured to guide the electromagnetic radiation generated by the one or more sources. The one or more sources may include one or more lasers, one or more Light Emitting Diodes (“LEDs”), one or more incandescent sources, and/or other electromagnetic radiation sources. The one or more optical elements may include one or more reflective elements, one or more refractive elements, one or more diffractive elements, and/or other optical elements that may be configured to guide electromagnetic radiation. The one or more optical elements may be actuable (e.g., via a microelectromechanical (“MEMS”) arrangement, via gyrating arrangement, etc.) to vary one or more aspects of the emission of the electromagnetic radiation generated by the one or more sources (e.g., to vary the directionality, pattern, etc.). In other implementations, the one or more sources themselves may be actuable to vary similar aspects of the emission of electromagnetic radiation from emission module 18.

In some implementations, emission module 18 may emit electromagnetic radiation with a dynamic spatial distribution, or pattern, that changes as time passes. This may include expanding and/or contracting the pattern as time passes. In some such implementations, emission module 18 may emit one or more beams of electromagnetic radiation that are scanned in a spiral pattern that expands and/or contracts as time passes. The spiral pattern may include a circular spiral pattern, a square spiral pattern, a triangular spiral pattern, and/or other spiral patterns. As another possibility, the dynamic pattern may comprise electromagnetic radiation having a cross-section of a predetermined shape that expands and/or contracts as time passes. For instance, emission module 18 may emit electromagnetic radiation with a cross-section of a circle, a square, a triangle, a cross, and/or other shapes that expand and/or contract as time passes. In some instances, the emission of electromagnetic radiation may be modulated (e.g., amplitude modulated, frequency modulated, etc.). The modulation rate (e.g., the frequency of the amplitude modulation) may be constant, or may be varied.

Emission module 18 may emit electromagnetic radiation over a solid angle that is relatively large (e.g., up to about

$$2 π ( 1 - \sqrt{3} / 2 )$$

steradians, up to about 2π steradians, etc.). Emitting the electromagnetic radiation over a relatively large solid angle may enable information related to the position and/or movement of tool 12 to be determined for an enhanced range of positions. For example, a relatively large solid angle of emission may enlarge the “foot print” on interface surface 32 of electromagnetic radiation emitted from tool 12 in instances in which tool 12 is positioned relatively close to interface surface 32. As another example, a relatively large solid angle of emission may enable interface surface 32 to receive electromagnetic radiation emitted from tool 12 in instances in which tool 12 is not pointed directly at interface surface 32.

Control module 20 may control emission module 18 to vary one or more aspects of the emission of electromagnetic radiation from emission module 18. Control module 20 may exercise control of emission module 18 to ensure that the electromagnetic radiation emitted by emission module 18 will enable information (e.g., positional information, movement information, biological function information, ambient conditions information, etc.) to be determined based at least in part by the detection of the emitted electromagnetic radiation by detection arrangement 14. Control module 20 may control emission module 18 to vary one or more aspects of the emission of electromagnetic radiation module 18 in accordance with an emission scheme. In some instances, the emission scheme may be constant, or “hard-wired,” within tool 12. In other instances, one or more aspects of the emission scheme may be set or changed by a user (e.g., by inputting information related to the emission scheme into system 10), by system 10 (e.g., to enable system 10 to distinguish between two or more tools), or otherwise set or changed.

Motion detection module 22 may be configured to detect information related to the position and/or motion of tool 12. For example, motion detection module 22 may include a gyroscope, an accelerometer, and/or other components capable of determining information related to the position and/or motion of tool 12. Motion detection module 22 may detect information related to, for instance, a velocity of tool 12, a distance and/or direction that tool 12 has been moved, an acceleration of tool 12, and/or other information related to the motion and/or position of tool 12.

Biological function module 24 may be configured to detect information related to one or more biological functions of a user interacting with tool 12. The one or more biological functions may include, for example, pulse, respiration, blood pressure, body temperature, perspiration, involuntary muscle actuation (e.g., shaking, startling, etc.), and/or other biological functions. Information related to one or more of biological functions monitored by biological function module 24 may be implemented to identify a user interacting with tool 12. For example, people generally have unique pulse signatures that are a result of the exact manner in which the heart muscle contracts and relaxes as blood is pumped, and the manner in which the pumping circulation of blood is modulated in the veins. The pulse signature of an individual may be affected by the size, shape, strength, and/or configurations of the chambers of individual's heart; the size and/or shape of the valves of the individual's heart; the cross-sectional size, length, and/or rigidity of the individual's veins; and/or other aspects of the individual's cardiovascular system. Based on a detection of the motion of tool 12 in the hands of a user, biological function module 24 may identify and/or quantify one or more aspects of the pulse signature of the user which may enable an identification of the user.

In some implementations, the information detected by biological function module 24 may be used as an input, and an electronic system (e.g., a gaming system) may be configured to receive input from the user via system 10 may adjust its interaction with the user based on the information detected by biological function module 24. For example, changes in pulse, respiration, blood pressure, body temperature, and/or perspiration may indicate a level of excitement of the user. Various aspects of a game being played by a user may then be enhanced or augmented based on the excitement level of the user. As another example, these types of bodily functions may indicate a fatigue level of the user that may trigger various effects in a game being played by a user. As yet another example, one or more of the bodily functions may be integrated into a game as a factor that the user must consider in order to be successful in the game. For instance, in a game in which the user is “shooting” (e.g., shooting a rifle, taking a picture with a camera, etc.), the user may be penalized for not taking a shot between heart palpitations (similar to the penalization in accuracy or stabilization that would be imposed in a real life situation).

Ambient conditions module 26 may be configured to detect information related to one or more ambient conditions in the environment in which tool 12 is being used. For instance, ambient conditions module 26 may be configured to detect information related to one or more of an ambient temperature, an ambient humidity, an altitude, an ambient pressure, and/or other ambient conditions. Information related to one or more ambient conditions detected by ambient conditions module 26 may be conveyed to a user. For instance, in implementations in which interface surface 32 is provided at the surface of an electronic display, such information may be conveyed to the user via interface surface 32. Such implementations may include, for example, electronic whiteboards, appliance control interfaces, and/or other implementations of system 10.

It should be appreciated that biological function module 24 and/or ambient conditions module 26, as described above, may include a vibration sensitive device, a temperature sensitive device (e.g., a thermometer, a thermocouple, etc.), a hygrometer, an altimeter, and/or other components capable of detecting information related to the biological functions and/or ambient conditions mentioned above. In some implementations, a single component may form part of both biological function module 24 and ambient conditions module 26. For example, a single temperature sensitive device may be utilized to detect information related to both a body temperature and ambient temperature.

Feedback module 28 may be configured to provide feedback from tool 12 to the user interacting with tool 12. For example, feedback module 28 may include one or more light sources (e.g., in addition to the one or more sources included in emission module 18), one or more audio speakers, one or more visual displays, one or more motion inducing systems designed to mechanically actuate tool 12 (e.g., a gyroscope that can “shake” tool 12), one or more electrodes capable of delivering an electrical current to the user, one or more heat dispersing elements, and/or other components capable of providing feedback to the user.

Communication module 30 may be configured to transmit information to and/or receive information from processor 16 by a medium other than the electromagnetic radiation emitted by emission module 18. The information may include control information provided to tool 12 from processor 16 and/or information detected by one or more of modules 22, 24, and/or 26. Information detected by one or more of modules 22, 24, and/or 26 may include, for example, one or more biological functions (e.g., obtained by biological function module 24 as discussed above), one or more ambient conditions (e.g., obtained by ambient condition module 26 as discussed above), supplemental information related to the position and/or motion of tool 12 (e.g., obtained by motion detection module 22). The communication between processor 16 and communication module 30 may be accomplished by a communication link that may include a wired connection, a network connection, a wireless connection, and/or other connections. In some implementations, as an alternative to communication via communication module 30, this same information may be communicated to processor 16 by varying one or more of the properties of the electromagnetic radiation emitted by tool 12. For example, tool 12 may vary a frequency, an amplitude, a frequency modulation, an amplitude modulation, a frequency, and/or other properties of the emitted electromagnetic radiation to communicate this information.

In some implementations, tool 12 may be a device that is designed specifically for implementation in system 10 without “external” functionality. However, in other embodiments, tool 12 may include devices that are useful in other contexts and are designed to include some or all of the functionality described herein with respect to tool 12 to enable them to be implemented for inputting information via system 10. For example, tool 12 may comprise a mobile telephone, a computer mouse, a display device, an audio device, a Person Digital Assistant (“PDA”), a camera, a microphone, and/or other devices. In some instances, tool 12 may include a leash, or tether, that may secure tool 12 to a user or a structure of some sort external to tool 12. For example, the leash may include a cord that attaches tool 12 to a band that can be secured to the wrist (or leg, or upper arm, etc.) of the user. Various implementations of tool 12 may include one or more detachable parts. For example, tool 12 may include a detachable racket/paddle head (e.g., a tennis racket head, a racquetball racket head, a badminton racket head, a ping pong paddle head, etc.), a detachable bat head (e.g., a baseball bat heat, a cricket bat head, etc.), a site or viewfinder that enables the user to site objects displayed on interface surface 32 (e.g., a gun site, a camera viewfinder, etc.), and/or other detachable components. A given detachable component may be merely a passive attachment, or it may include one or more active elements, the passive and/or active elements of the given detachable component may be designed to enhance the users interaction with an electronic system. For instance, an attachment may include one or more gyros designed to be driven to provide feedback to the user (e.g., a racket/paddle or bat head), an attachment may provide optics that enhance the users interaction (e.g., optics of a gun site or viewfinder), and/or an attachment may be configured to enhance the users interaction with the electronic system in other ways.

As was mentioned above, in some embodiments, detection arrangement 14 may be configured to receive electromagnetic radiation emitted by tool 12, and to generate one or more output signals based on one or more properties of the received electromagnetic radiation. The one or more properties of the received electromagnetic radiation upon which the one or more output signals are based may include the location(s) at which the electromagnetic radiation becomes incident on detection arrangement 14, intensity, frequency, amplitude modulation, frequency modulation, direction of propagation, and/or other properties. In some implementations, detection arrangement 14 may provide an interface surface 32, and the one or more output signals may be related to the location on interface surface 32 at which the electromagnetic radiation emitted by tool 12 becomes incident.

For instance, detection arrangement 14 may include an optical touchpad that provides interface surface 32. Some suitable examples of an optical touchpad are discussed in U.S. patent application Ser. No. 10/507,018, entitled “Touch Pad, A Stylus for Use With the Touch Pad, and A Method of Operating the Touch Pad,” and filed Mar. 21, 2005; U.S. patent application Ser. No. 10/548,625, entitled “TITLE,” and filed FILING DATE; U.S. patent application Ser. No. 10/571,561, entitled “TITLE,” and filed FILING DATE; U.S. patent application Ser. No. 10/548,664, entitled “System and A Method of Determining the Position of a Radiation Emitting Element,” and filed Mar. 12, 2004; U.S. Provisional Patent Application No. 60/787,164, entitled “TITLE,” and filed FILING DATE; International Patent Application No. PCT/DK2004/00596, entitled “A system and Method of Determining A Position of A Radiation Emitting Element,” and filed Sep. 9, 2004; U.S. patent application Ser. No. 11/320,742, entitled “Optical Touchpad With Multilayer Waveguide,” and filed Apr. 5, 2006; U.S. patent application Ser. No. 11/480,865, entitled “Optical Touchpad System and Waveguide for Use Therein,” and filed Jul. 6, 2006; U.S. patent application Ser. No. 11/480,892, entitled “Optical Touchpad System and Waveguide for Use Therein,” and filed Jul. 6, 2006; U.S. patent application Ser. No. 11/480,893, entitled “Optical Touchpad With Three-Dimensional Position Determination,” and filed Jul. 6, 2006; and U.S. patent application Ser. No. 11/581,126, entitled “Interactive Display System, Tool for Use Therein, and Tool Management Apparatus,” and filed Oct. 16, 2006 (“the touchpad applications”). These applications are hereby incorporated by reference into this disclosure in their entirety. As is discussed in, for example, the touchpad applications, the optical touchpad may include a waveguide optically coupled to one or more electromagnetic radiation detectors. The waveguide may include a waveguide layer, sometimes called an “underlayer” or “signal layer,” capable of guiding electromagnetic radiation that is incident on the interface surface of the optical touchpad to the one or more electromagnetic radiation detectors by total internal reflection. The electromagnetic radiation detectors may then generate one or more output signals based on the electromagnetic radiation received from the waveguide layer.

The implementation of alternative optical touchpads capable of generating one or more output signals in response to receiving electromagnetic radiation from tool 12 is also contemplated. For instance, the optical touchpad may include one or more radiation sensitive pixels (e.g., implementing thin-film transistor (“TFT”) technology) that, through inherent photo current properties, form electromagnetic radiation detectors in the interface surface provided by the optical touchpad. Other optical touchpads are also contemplated.

In some embodiments, detection arrangement 14 may include an array of electromagnetic radiation detectors arranged at the perimeter of interface surface 32. In these embodiments, the electromagnetic radiation detectors in the array may generate output signals that indicate if electromagnetic radiation is being received by a given electromagnetic radiation detector directly from tool 12. The output signals may further be related to one or more properties of the incident electromagnetic radiation.

According to various embodiments of the invention, processor 16 may be operatively coupled with detection arrangement 14. The operative coupling may be accomplished via a communication link that includes a wired link and/or a wireless link. Over this communication link, information may be exchanged between detection arrangement 14 and processor 16. For instance, the one or more output signals generated by detection arrangement 14 (and/or information derived therefrom) may be provided to processor 16 over the communication link.

It should be appreciated that although processor 16 is shown in FIG. 1 as a single entity, this is for illustrative purposes only. In some implementations, processor 16 may include a plurality of processing units. These processing units may be physically located within the same device, or processor 16 may represent processing functionality of a plurality of devices operating in coordination. In instances in which a plurality of devices are implemented, operative communications links may be formed between the devices to enable communication and coordination therebetween. For example, in some embodiments, processor 16 may include one or more processors external to the other components of system 10 (e.g., a host computer), one or more processors that are included integrally in one or more of the components of system 10 (e.g., a processor included integrally with detection arrangement 14, a processor included integrally with tool 12, etc.), or both. Processors external to other components within system 10 may, in some cases, provide redundant processing to the processors that are integrated with components in system 10, and/or the external processor may provide additional processing to determine additional information.

As is shown in FIG. 1, processor 16 may include surface position module 34, a pattern module 36, a tool position module 38, a biological function module 40, a tool coordination module 42, and/or other modules. Modules 34, 36, 38, 40, and/or 42 may be implemented in software; hardware; firmware; some combination of software, hardware, and/or firmware; and/or otherwise implemented. It should be appreciated that although modules 34, 36, 38, 40, and 42 are illustrated in FIG. 1 as being co-located within a single processing unit, in implementations in which processor 16 includes multiple processing units, modules 34, 36, 38, 40, and/or 42 may be located remotely from the other modules and operative communication between modules 34, 36, 38, 40, and/or 42 may be achieved via one or more communication links. Such communication links may be wireless or hard wired.

Surface position module 34 may determine the location(s) on and/or near interface surface 32 of detection arrangement 14 where electromagnetic radiation is received from tool 12. This determination may be made using conventional methods for determining such information. For example, in implementations that include an optical touchpad similar to one of the optical touchpads described in one or more of the touchpad applications, the determination may be made based on one or more properties of the electromagnetic radiation that are received by detection arrangement 14 from tool 12. As is described in the touchpad applications, the one or more properties may include a location of incidence, a direction of propagation at interface surface 32 and/or within a waveguide associated with detection arrangement 14, relative intensity, and/or other properties. As another example, in either implementations that include a display with electromagnetic radiation sensitive pixels, or in implementations that include an array of electromagnetic radiation detectors arranged at the periphery of interface surface 32, the pixels or detectors on which radiation is incident may be determined based on the intensity of electromagnetic radiation received by the pixels or detectors. In some implementations including the display with electromagnetic radiation sensitive pixels, the pixels may be read-out together similar to the manner in which an imaging chip (e.g., a CMOS chip, a CCD chip, etc.) is read-out to provide a “snapshot” of the incident radiation at a given point in time.

In addition to the location(s) on and/or near interface surface 32 at which electromagnetic radiation is received from tool 12, surface position module 34 may determine information related to one or more properties of the incident electromagnetic radiation. For example, surface position module 34 may determine information related to intensity, frequency, and/or other properties of the electromagnetic radiation.

Pattern module 36 may analyze the determinations made by surface position module 34 (e.g., the position on interface surface 32 at which the electromagnetic radiation was incident, the frequency of the electromagnetic radiation, the intensity of the electromagnetic radiation, etc.), and from these determinations may identify information related to the pattern of electromagnetic radiation emitted by tool 12. This may include distinguishing between electromagnetic radiation received at interface surface 32 from tool 12 and other similar tools. Such electromagnetic radiation may be distinguished based on the intensity, modulation (e.g., frequency modulation, amplitude modulation, etc.), frequency, spatial distribution (e.g., the general shape of the emitted pattern) and/or other properties of the electromagnetic radiation. In analyzing the determinations made by surface position module 34, pattern module 36 may determine information related to the pattern of the electromagnetic radiation that is received at or near interface surface 32 from tool 12. This information may include, for example, sizes and/or shapes of areas or zones at or near interface surface 32 that receive electromagnetic radiation, the shape and/or timing of a spatial and/or temporal pattern formed by the received electromagnetic radiation, and/or other information. Various aspects of this determination are discussed below.

Based on the information determined by pattern module 36, tool position module 38 may determine information related to the position and/or the motion of tool 12. The information and/or the motion of tools 12 determined by tool position module 38 may include, for example, information related to a location at or near interface surface 32 at which tool 12 is pointed, the position of tool 12 in three dimensions with respect to interface surface 32, the position of tool 12 in three rotational degrees of freedom (e.g., the roll of tool 12, the yaw of tool 12, the pitch of tool 12, etc.), and/or other information related to the position of tool 12. In some instances, this information may be determined based on the one or more output signals generated by detection arrangement 14 in response to reception of electromagnetic radiation emitted by tool 12. Various aspects of these types of determination are discussed below.

In some implementations, the determination of information related to the position and/or motion of tool 12 made by tool position module 38 based on the one or more output signals generated by detection arrangement 14 may be supplemented by information detected by motion detection module 22. For instance, determinations of information related to the position and/or motion of tool 12 by tool position module based on the one or more output signals from detection arrangement 14 may suffer from lapses during which tool 12 is pointed by the user to a point not on or near interface surface 32 of detection arrangement 14 such that effectively none of the electromagnetic radiation emitted by tool 12 becomes incident on detection arrangement 14. However, information obtained by motion detection module 22 (e.g., a gyroscope, an accelerometer, etc.) related to the position and/or motion of tool 12 may be used to fill in these lapses, thereby bridging the gaps in time during which tool 12 is not pointed to a point at or near interface surface 32.

Biological function module 40 may determine information related to one or more biological functions of a user interacting with tool 12. For example, based on fluctuations in the position of tool 12 (as determined by tool position module 38), biological function module 40 may determine information related to pulse, involuntary muscle actuation, and/or other biological functions. In some implementations, the information detected by biological function module 40 may be used as to identify a user and/or to adjust an interaction between an electronic system and the user (e.g., as was described above with respect to biological function module 24).

Tool coordination module 42 may communicate with tool 12 (e.g., via communication module 30) to coordinate the implementation of tool 12 with a plurality of other tools being used to input information to system 10. Tool coordination module 42 may communicate with the various tools being implemented (e.g., tool 12) to ensure that the electromagnetic radiation being emitted by the various tools will be distinguishable by processor 14 based on the one or more output signals generated by detection arrangement 14. For example, tool coordination module 42 may communicate with the tools to ensure that each tool is emitting electromagnetic radiation with a unique intensity, spatial distribution, modulation, shape, and/or combination thereof. Tool coordination module 42 may coordinate these and/or other aspects of the emission of electromagnetic radiation by the individual tools to pattern module 36 to enable pattern module 36 to distinguish the electromagnetic radiation emitted by tool 12 from electromagnetic radiation emitted by other tools.

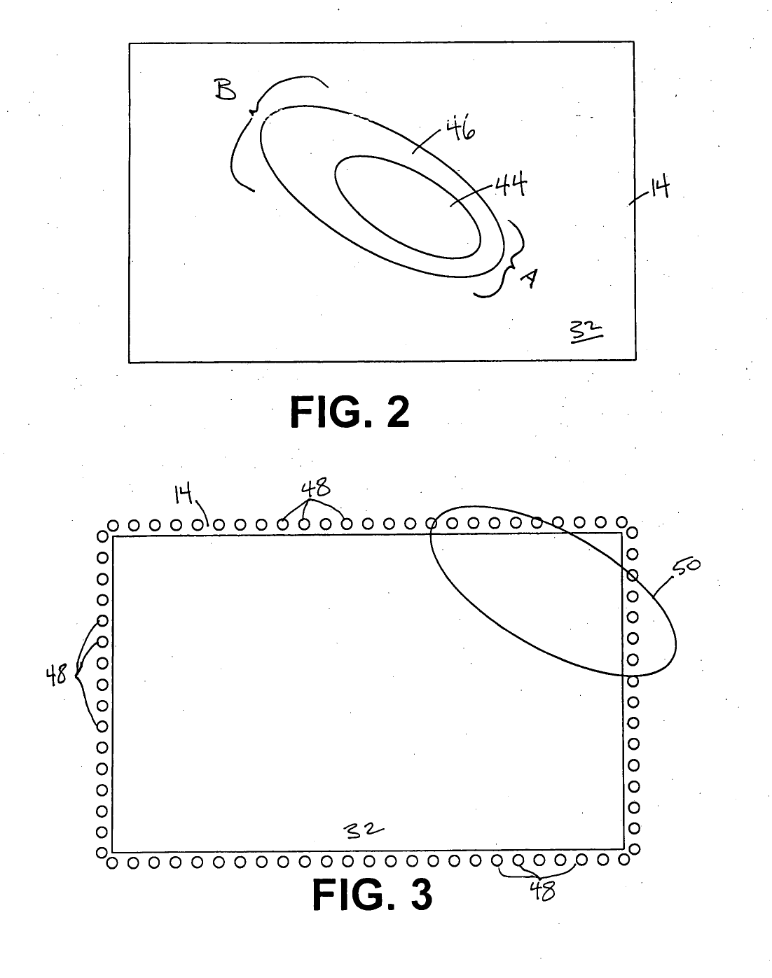

FIG. 2 illustrates interface surface 32 of detection arrangement, according to one or more embodiments of the invention. More particularly, FIG. 2 illustrates interface surface 32 in embodiments in which tool 12 emits a cone of electromagnetic radiation. The cone of electromagnetic radiation emitted by tool 12 may expand and retract with time. For example, at a first point in time, the electromagnetic radiation emitted by tool 12 becomes incident on interface surface 32 in a first zone 44, while the electromagnetic radiation emitted by tool 12 becomes incident on a second zone 46 at a second point in time. The expansion of the zone of illumination on interface surface 32 from first zone 44 to second zone 46 may be a continuous expansion. However, in some other implementations, the emission of electromagnetic radiation by tool 12 may be amplitude modulated to provide pulses of electromagnetic radiation. For instance, a first pulse of electromagnetic radiation may become incident on interface surface 32 at first zone 44 and a second pulse of electromagnetic radiation may become incident on interface surface 32 at second zone 46. In other implementations, the emission of electromagnetic radiation by tool 12 may be frequency modulated. For example, electromagnetic radiation of a first frequency may be emitted as the zone of illumination expands from first zone 44 to second zone 46, at which time the frequency of the radiation is changed to a second frequency. This change in frequency may be detected to determine the difference in size between first zone 44 and second zone 46. In still other implementations, one or more other properties of the electromagnetic radiation may be modulated in a similar fashion. It should be appreciated that hereafter as various aspects of system 10 are described with respect to implementations in which pulses of radiation are emitted as tool 12 modulates the amplitude of the emitted electromagnetic radiation, the description of amplitude modulation is not intended to be limiting. In the described implementations, one or more properties of the emitted electromagnetic radiation other than amplitude may be modulated in place of amplitude modulation without departing from the scope of this disclosure.

By determining the center of zones 44 and 46, the point on interface surface 32 at which the user was pointing tool 12 at the first and second points in time may be determined. The determination of the center of the zones of electromagnetic radiation (e.g., zones 44 and 46) on interface surface 32 may be made by tool position module 38. The determination of the point on interface surface 32 at which the user was pointing may be used to input information by the user into system 10. For example, the user may make a selection by pointing to a specific area on interface surface 32. As another example, the user may point tool 12 to an area on interface surface 32 to interact with a virtual object being displayed as part of a game (e.g., to shoot or hit the object). Other information may also be input in this manner.

It should be appreciated that one or more aspects of the shape and size of the first and second zones 44 and 46 are a function of the position of tool 12 with respect to interface surface 32. For example, it should be appreciated that unless tool 12 emits the electromagnetic radiation along an optical axis that is perpendicular to interface surface 32, zones 44 and 46 may not represent a cross-section of the pattern of emitted electromagnetic radiation. Instead, zones 44 and 46 may be elongated (e.g., from a circular cross-section to an elliptical zone of illumination on interface surface 32) in a direction that corresponds to a directional orientation of an angle between the optical axis along which the electromagnetic radiation is emitted and an axis that is perpendicular to interface surface 32 (e.g., this angle accounts for the pitch and yaw of tool 12 with respect to the perpendicular to interface surface 32.). The amount of elongation of zones 44 and 46 corresponds to the magnitude of the angle (e.g., the larger the angle, the more elongated zones 44 and 46 may become). Accordingly, based on the deformation of the zones of electromagnetic radiation formed on interface surface 32, the direction from which the electromagnetic radiation has emanated (e.g., tool 12) may be determined. This calculation may be performed, for example, by tool position module 38 based at least in part on the shape formed by the zone(s) on interface surface 32 determined to have received electromagnetic radiation by surface position module 34.

Thus, based on the position and shape of either of zones 44 or 46 the (i) location at or near interface surface 32 that the user is pointing tool 12, and (ii) the direction of tool 12 with respect to interface surface 32 can be determined. By analyzing the change in size of the area of interface surface 32 over time (e.g., from zone 44 at the first point in time to zone 46 at the second point in time), the distance from interface surface 32 to tool 12 may be determined (e.g., by tool position module 38). It should be appreciated that if the field of emission of the electromagnetic radiation emitted by tool 12 is varied (e.g., contracted or expanded) as a function of time in a predetermined manner (e.g., at a predetermined rate), corresponding changes in the size of the zones on interface surface 32 receiving the electromagnetic radiation will become larger as tool 12 is moved away from interface surface 32. Similarly, as tool 12 is moved toward interface surface 32, the changes in size of the zones on interface surface 32 receiving electromagnetic radiation will become smaller. Provided that the function being implemented by tool 12 to vary the size of the field of emission (e.g., the rate of expansion and/or contraction), this relationship may be leveraged to determine the distance of tool 12 to interface surface 32. This calculation may be made, for example, by tool position module 38 based on the information determined by surface position module 34 and the function used by tool 12 to vary the size of the filed of emission (e.g., determined and/or stored by tool coordination module 42).

Upon determination of the distance of tool 12 from interface surface 32, the position of tool 12 in three dimensions may be determined (e.g., the determined distance along the determined optical axis from the center of the illuminated zone on interface surface 32). Further, the orientation of tool 12 in two degrees of freedom may be determined (based on the orientation of the optical axis). This determination may be referred to as the “tilt” of tool 12 with respect to interface surface 32.

In some instances where the electromagnetic radiation emitted from tool 12 is pulsed, the determination of the distance between tool 12 and interface surface 32, and/or the determination of the tilt of tool 12 may be made (or refined) based on the spatial differences between the zones on interface surface 32 illuminated by temporally proximate pulses of electromagnetic radiation emitted from tool 12. For example, due to the tilt of tool 12, the expansion of the electromagnetic radiation from first zone 44 to second zone 46 will be skewed such that the boundary of the illuminated zone at areas closer to tool 12 (e.g. illustrated as region A in FIG. 2) may be slower than for areas relatively further from tool 12 (e.g., illustrated as region B in FIG. 2). Further, the greater the magnitude of the tilt of tool 12, the larger this relative difference may become. Thus, based on the general expansion of the electromagnetic radiation between pulses (e.g., the overall increase in area) between pulses, the distance between tool 12 and interface surface 32 may be determined (or the determination may be refined). And, based on the relative difference in expansion of the boundary of the illumination zone between pulses, the direction from interface 32 to tool 12 (or the tilt of tool 12) may be determined (or the determination may be refined).

In some embodiments, the rotational orientation of tool 12 about the optical axis (e.g., also referred to as the “roll” of tool 12) may further be determined. To enable this determination, the field of emission of tool 12 may be marked in some way. For example, an irregularity may be provided at one location on the boundary of the field (e.g., a protrusion, an intrusion, etc.), or within the field (e.g., a “hole), that may be identified in the corresponding zone created on interface surface 32. As another example, the electromagnetic radiation emitted by tool 12 may be filtered in such a way as to mark the electromagnetic radiation. For instance, electromagnetic radiation in one area of the field of emission may be provided with a different frequency, intensity, and/or modulation than other areas of the field. Other mechanisms for marking the electromagnetic radiation emitted by tool 12 may be employed.

Based on the orientation of the marked area of the zone formed on or near interface surface 32 by the electromagnetic radiation emitted by tool 12, tool position module 38 may determine the rotational orientation of tool 12 about the optical axis of the emitted electromagnetic radiation. This determination, in conjunction with the other determinations, discussed above, related to the position of tool 12 with respect to interface surface 32 may enable tool position module 38 to determine the position of tool 12 in six degrees of freedom (e.g., three translational degrees of freedom and three rotational degrees of freedom). Some or all of this positional information may be used to input information to input system 10. For example, the information may be used in a gaming environment to control a subject in an electronic game. As another example, a display of information may be moved in coordination with changes in position of tool 12.

Processor 16 (e.g., tool position module 38) may implement the determined position information to determine information related to motion of tool 12 by the user. For example, determinations of position may be aggregated to determine time derivatives of the position of tool 12 such as velocity, acceleration, jerk, etc. These time derivatives may be determined to describe translational and/or rotational motion. Such aggregations of position information may be achieved using conventional mechanisms for determining time derivatives of position. These values (velocity, acceleration, jerk, etc.) may also be used as a mechanism for enabling the user to input information to input system 10.

As was mentioned above, in some embodiments of the invention, detection arrangement 14 may include an array of electromagnetic radiation detectors arranged at the periphery of interface surface 32 to receive electromagnetic radiation directly from tool 12 (e.g., not through a waveguide layer). FIG. 3 illustrates detection arrangement 14 including such an array of electromagnetic radiation detectors 48, according to one or more embodiments of the invention. As can be seen in FIG. 3, in these embodiments, the one or more output signals generated by detection arrangement 14 may correspond to one or more properties of electromagnetic radiation received at the periphery of interface surface 32, rather than electromagnetic radiation received incident directly onto interface surface 32.

For instance, in FIG. 3, the output signal(s) generated by electromagnetic radiation detectors 48 in response to electromagnetic radiation emitted by tool 12 (illustrated in FIG. 3 as emission zone 50) may indicate which ones of electromagnetic radiation detectors 48 receive the emitted electromagnetic radiation. If the general shape of the emission field of tool 12 is known (e.g., by processor 16), then zone 50 on interface surface 32 formed by electromagnetic radiation emitted from tool 12 at a given point in time may be determined based on which ones of electromagnetic radiation detectors 48 received electromagnetic radiation from tool 12 at the given point in time. From this determination, calculations to derive information related to the position and/or motion of tool 12 may follow as described above.

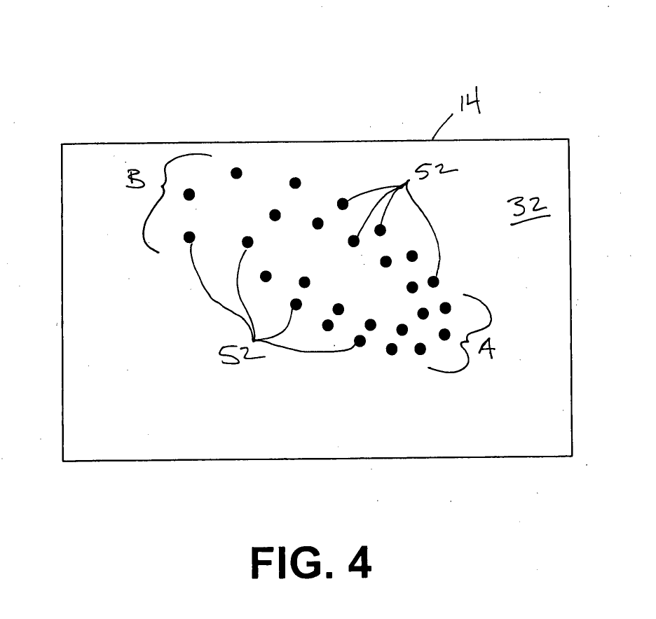

FIG. 4 illustrates an alternative emission scheme that may be employed to emit electromagnetic radiation from tool 12 to enable position, motion, and/or other information related to tool 12 to be determined, according to one or more embodiments of the invention. In the emission pattern illustrated in FIG. 4, rather than emitting electromagnetic radiation in an emission field with a predetermined shape that expands and/or contracts as a function of time, tool 12 may scan a beam of electromagnetic radiation in a predetermined pattern. The predetermined pattern may expand and/or contract with time. For instance, the beam may be scanned in a spiral pattern, such as a circular spiral pattern, a triangular spiral pattern, a square spiral pattern, and/or other differently shaped spiral patterns.

In the illustration of an emission pattern provided in FIG. 4, the beam is scanned by tool 12 in a circular spiral pattern, and is further pulsed (e.g., amplitude modulated) to provide pulses of electromagnetic radiation in a circular spiral pattern that expands and/or contracts with time. In some instances, the pulse rate (e.g., the frequency of the amplitude modulation) may be constant over time. In other instances, the pulse rate may also be varied with time, and may even be random. It should be appreciated that in instances described herein in which a beam is scanned in a pattern (e.g., a spiral pattern) that is expanded and/or contracted over time, tool 12 may actually emit and scan a plurality of beams. In fact, this may provide redundancy to calculations related to the position and/or motion of tool 12.

As the beam of electromagnetic radiation is scanned and pulsed by tool 12, a series of illumination zones 52 are created on or near interface surface 32 by the emitted electromagnetic radiation. Based on the one or more output signals generated by detection arrangement 14 in response to receiving electromagnetic radiation in illumination zones 52, processor 16 may determine the position of one or more of illumination zones 52 on interface surface 32 (e.g., by surface position module 34). This determination may enable processor 16 to determine the shape and location of the pattern that the beam is being scanned in by tool 12 (e.g., by pattern module 36). Once the shape and location of the pattern on or near interface surface 32 is determined, processor 16 may determine other information related to the position and/or movement of tool 12 (e.g., by tool position module 38). For example, in the circular spiral pattern illustrated in FIG. 4, processor 16 (e.g., tool position module 38) may implement calculations similar to the calculations described above with respect to the conical emission scheme of FIG. 2 (e.g., the circles of the spiral correspond to the circular cross-section of the cone) to determine information such as the location on or near interface surface 32 at which tool 12 is being pointed by the user (e.g., the center of the pattern), the direction of tool 12 with respect to interface surface 32 (e.g., based on the elongation of the pattern), and/or the distance between tool 12 and interface surface 32 (e.g., based on the rate of expansion/contraction of the pattern).

In some implementations, the spatial differences between proximally illumination zones 52 may be analyzed to determine (or refine determinations of) the distance between interface surface 32 and/or the tilt of tool 12 with respect to interface surface 32. For example, as was discussed above with respect to FIG. 2, for areas of the spiral pattern formed by illumination zones 52 that are closer to tool 12 (e.g., illustrated in FIG. 4 as area A), the distance between zones 52 formed by successive pulses will be smaller than the distance between zones 52 formed by successive pulses in areas that are further away from tool 12 (e.g., illustrated in FIG. 4 as area B). Thus, by analyzing the respective spatial separation of zones 52 in different areas of the spiral, the determination of the direction from interface surface 32 to tool 12 may be made (or the determination may be refined). Further, the distance between zones 52 formed by successive pulses may also be impacted by the distance from tool 12 to interface surface 32. Accordingly, based on an overall trend in the distances between zones 52 (e.g., an average distance for one “circuit” around the spiral), the distance between tool 12 and interface surface 32 may be determined (or the determination may be refined).

In implementations in which the beam of electromagnetic radiation is pulsed by tool 12, various properties of illumination zones 52 formed by the pulses of electromagnetic radiation emitted by tool 12 may further be used to determine additional information and/or refine the determinations enumerated above. For example, rather than relying on spatial and/or frequency differentiation to mark the pattern emitted by tool 12 to enable determination of the roll of tool 12 (as discussed above with respect to FIG. 2), the pattern may be marked by varying the chirp rate at different portions of the pattern. Marking the pattern in this manner may enable processor 16 to determine the roll of tool 12 by determining the rotational orientation of tool 12 about an axis running from tool 12 to interface surface 32. Emitting electromagnetic radiation from tool 12 as one or more beams that are scanned according to a predetermined pattern, and chirping the beam(s) may further reduce the overall photon budget of system 10, reduce the power consumption of system 10, and/or provide other enhancements.

It should be appreciated that implementations of tool 12 in which electromagnetic radiation is emitted as a beam that is scanned according to a predetermined pattern may also be employed with implementations of detection arrangement 14 in which detection arrangement 14 includes an array of electromagnetic radiation detectors arranged at or near the periphery of interface surface 32 (e.g., as shown in FIG. 3). As is described with respect to FIG. 3, in these implementations information related to the pattern of emission that is incident on interface surface 32 may be extrapolated from electromagnetic radiation in pulses emitted by tool 12 that become directly incident on one or more of the electromagnetic radiation detectors arranged at or near the periphery of interface surface 32. From the extrapolated information related to the pattern of emission incident on interface surface 32, information related to the position and/or motion of tool 12 may be determined.

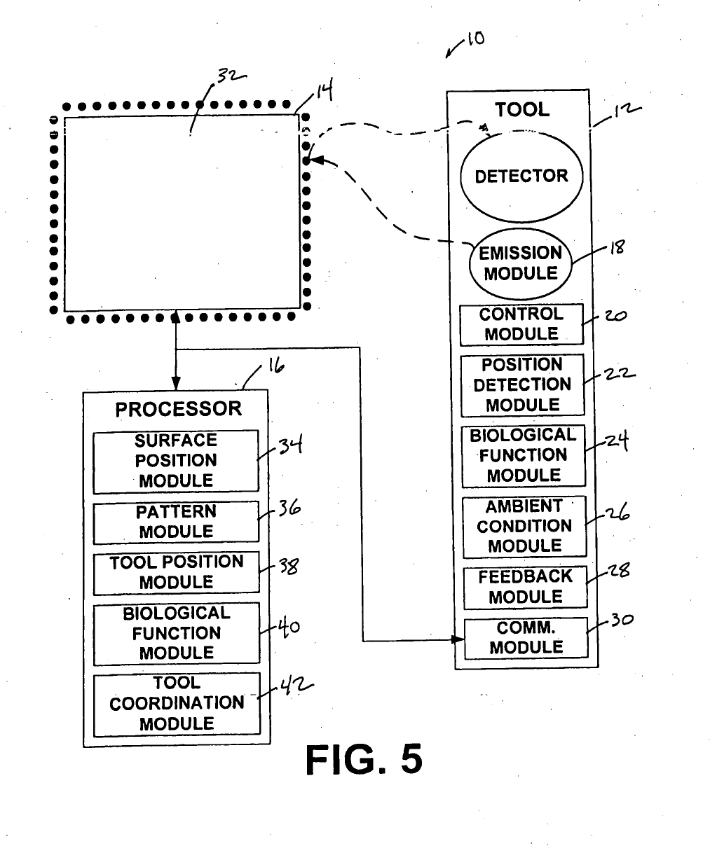

FIG. 5 illustrates an alternative configuration of detection arrangement 14, according to one or more embodiments of the invention. In the configuration of detection arrangement 14 illustrated in FIG. 5, detection arrangement 14 includes one or more electromagnetic radiation detectors 54 carried on tool 12 and one or more reflectors 56 provided at or near interface surface 32. Reflectors 56 may include one or more retroreflectors configured to reflect at least a portion of the electromagnetic radiation emitted by tool 12 from interface surface 32 back toward tool 12. In some implementations, reflectors 56 may include an array of reflectors positioned at or near the periphery of interface surface (e.g., similar to the positioning of electromagnetic radiation detectors 48 in FIG. 3). In some implementations, reflectors 56 may be integrated into interface surface 32. In these implementations, reflectors 56 may be provided within interface surface according to a predetermined distribution. The predetermined distribution may include a predetermined spacing (which may be constant, or may vary based on position on interface surface 32), a predetermined density, a predetermined distribution pattern, etc. Reflectors 56 may be applied to interface surface 32 to retrofit system 10 to an existing display or surface. For example, reflectors 56 may be provided at the periphery of interface surface 32 without disrupting the display of information on interface surface 32. As another example, reflectors 56 may be integrated into a film or coating that may be applied to interface surface 32. The film or coating may be formed to be substantially transparent with respect to electromagnetic radiation passing through interface surface 32 toward the user, but may be reflective (or include reflective portions) for electromagnetic radiation of the frequency emitted by tool 12 that become incident on interface surface 32 from the direction of the user.

Electromagnetic radiation detectors 54 may include an array of one or more photosensitive elements that generate the one or more output signals in response to received electromagnetic radiation. For example, electromagnetic radiation detectors 54 may include an array of photodiodes (e.g., a single photodiode, an avalanche photodiode, an organic electronic photodiode, etc.), a CMOS array, a CCD array, or another array of photosensitive elements. The one or more output signals may enable the array formed by electromagnetic radiation detectors 54 to be “read out” as an image of an area at which tool 12 is pointed.

As tool 12 emits electromagnetic radiation toward interface surface according to a pattern that varies as a function time (e.g., as discussed above), the electromagnetic radiation reflected by reflectors 56 back towards tool 12 may become incident on electromagnetic radiation detector(s) 54. Based on the output signal(s) generated by electromagnetic radiation detector(s) 54, processor 16 may determine information related to the position and/or motion of tool 12. For example, based on the output signals(s) generated by electromagnetic radiation detector(s) 54, processor 16 may determine information related to the position of one or more zones of electromagnetic radiation on interface surface 32 (e.g., by surface position module 34). This information may include the position of the one or more zones, the shape of the one or more zones, temporal relationships between the one or more zones, etc.

In order to determine the information enumerated above, processor 16 may first determine the location(s) on or near interface surface 32 from which electromagnetic radiation emitted by tool 12 is being reflected. This may include analyzing an image of the area at which tool 12 is being pointed. By comparing a position at which the image indicates that electromagnetic radiation emitted by tool 12 has been reflected with one or more positions indicated by the image to include orientation marks provided at or near interface surface 32. These orientation marks may include features that are fixedly provided to predetermined locations at or near interface surface 32. The orientation marks may include one or more areas that are darker (e.g., more absorptive) or lighter (e.g., more reflective). By comparing one or more positions in an image of interface surface 32 indicating a reflection of electromagnetic radiation by one of reflectors 56 with the predetermined of the orientation mark(s) in the image, the position of the one or more reflectors 56 indicated in the image as reflecting electromagnetic radiation with respect to interface surface 32 may be determined. From this information (and the known pattern of emission of tool 12), the zones of electromagnetic radiation on interface surface 32 created by the electromagnetic radiation emitted by tool 12 may be extrapolated (e.g., by pattern module 36 in the manner discussed above with respect to FIGS. 2-4).

Once the zones on or near interface surface 32 that receive electromagnetic radiation from tool 12 are determined, information related to the position and/or motion of tool 12 with respect to interface surface 32 may be determined. For example, as is discussed above, a location at or near interface surface 32 to which tool 12 is being pointed, a direction from such a point to tool 12, a distance between interface surface 32 and tool 12, and/or other information related to the position and/or motion of tool 12 may be determined from the zones on or near interface surface 32 that receive electromagnetic radiation from tool 12.

It should be appreciated that in implementations of system 10 in which detection arrangement 14 includes one or more electromagnetic radiation detectors 54 carried on tool 12, that some or all of the functionality of processor 16 may also be included integrally with tool 12. For example, one or more of surface position module 34, pattern module 36, and/or tool position module 38 may be provided on tool 12 to enable an actual determination of the information related to the position and/or motion of tool 12 with respect to interface surface 32 to be made at tool 12. In such implementations, communications module 30 may communicate the determined information to an electronic system (e.g., a gaming system, an information management system, etc.) operatively linked to system 10 to enable the electronic system to use the determined information as input from the user interacting with tool 12.

The functionality of processor 16 provided within tool 12 may include, in some instance, the functionality of tool coordination module 42. For example, in implementations in which system 10 includes a plurality of tools similar to tool 12, the tools may communicate amongst each other (e.g., via communications module 30) to ensure that the electromagnetic radiation emitted by each tool will be distinguishable from the electromagnetic radiation emitted by the other tools. In such implementations, one of the tools may be designated as the “master” tool, and the other tools may be designated as “slave” tools. The master tool may provide instructions to the slave tools to provide coordination to the tools.

What is claimed is:

1. A system comprising:
a tool configured to emit electromagnetic radiation, wherein one or more aspects of the emission of electromagnetic radiation by the tool varies as a function of time;
a detection arrangement configured to receive electromagnetic radiation emitted by the tool and to generate one or more output signals based on one or more properties of the received electromagnetic radiation; and
a processor configured to receive the one or more output signals generated by the detection arrangement and to determine the position of the tool with respect to the detection arrangement based at least in part on the received one or more output signals.

2. The system of claim 1, wherein the tool is configured such that the one or more aspects of the emission of electromagnetic radiation that are varied as a function of time comprise one or both of an amplitude of the electromagnetic radiation and a direction of the emission of electromagnetic radiation with respect to the tool.

3. The system of claim 1, wherein the tool further comprises a biological function module configured to detect information related to one or more biological functions of a user interacting with the tool, and wherein the tool is further configured to adjust one or more aspects of the emission of electromagnetic radiation based on the information related to the one or more biological functions that is detected by the biological function module.

4. The system of claim 3, wherein the one or more biological functions comprise one or more of pulse, respiration, blood pressure, body temperature, perspiration, or involuntary muscle actuation.

5. The system of claim 1, wherein the one or more properties of the received electromagnetic radiation upon which the generation of the one or more output signals is based comprises one or more of an intensity of the electromagnetic radiation, a frequency of the electromagnetic radiation, an amplitude modulation of the electromagnetic radiation, a frequency modulation of the electromagnetic radiation, or a direction of propagation of the electromagnetic radiation.

6. The system of claim 1, wherein the one or more output signals generated by the detection arrangement in response to a given portion of the electromagnetic radiation received by the detection arrangement from the tool is indicative of a location on the detection arrangement at which the given portion of the electromagnetic radiation was incident.

7. The system of claim 1, wherein the one or more aspects of the emission of electromagnetic radiation by the tool are varied as a function of time in a predetermined manner.

8. The system of claim 1, wherein the processor is configured to determine the three dimensional position of the tool with respect to the detection arrangement and information related to one or more biological functions of a user interacting with the tool based at least in part on the received one or more output signals.

9. The system of claim 8, wherein the one or more biological functions comprise one or more of pulse, respiration, blood pressure, body temperature, perspiration, or involuntary muscle actuation.

10. The system of claim 1, wherein the detection arrangement comprises one or more reflectors positioned remotely from the tool and one or more radiation detectors carried on the tool such that at least a portion of the electromagnetic radiation received by the detection arrangement at the one or more reflectors is reflected back to the one or more radiation detectors carried on the tool.

11. The system of claim 1, wherein the detection arrangement comprises waveguide and one or more radiation detectors, and wherein the waveguide includes a waveguide layer that is configured to direct at least a portion of the received electromagnetic radiation to the one or more radiation detectors by total internal reflection.

12. The system of claim 1, wherein the detection arrangement comprises a pixilated display having one or more pixels capable of detecting electromagnetic radiation incident thereon.

13. The system of claim 1, wherein the processor is configured to determine the position of the tool with respect to the detection arrangement in three dimensions.

14. The system of claim 1, wherein the processor is configured to determine the position of the tool with respect to the detection arrangement in six degrees of freedom.

15. A tool for implementation in an input system capable of detecting the three dimensional position of the tool, the tool comprising:
an emission module configured to emit electromagnetic radiation therefrom such that one or more aspects of the emission of the electromagnetic radiation can be varied;
a control module configured to control the emission module to vary one or more aspects of the emission of the electromagnetic radiation as a function of time.

16. The tool of claim 15, wherein the control module is configured to control the emission module to vary one or more of the directionality of the emitted electromagnetic radiation, the amplitude of the emitted electromagnetic radiation, the frequency of the emitted electromagnetic radiation, the amplitude modulation of the emitted electromagnetic radiation, or the frequency modulation of the emitted electromagnetic radiation.

17. The tool of claim 15, wherein the control module is configured to control the emission module to emit the electromagnetic radiation in a predetermined pattern that expands and/or contracts over time.

18. The tool of claim 15, further comprising a motion detection module that determines information related to the motion of the tool in at least two dimensions.

19. The tool of claim 18, wherein the motion detection module comprises one or both of a gyroscope and an accelerometer.

20. The tool of claim 15, further comprising a biological function module that is configured to detect information related to one or more biological functions of a user interacting with the tool.

21. The tool of claim 20, wherein the one or more biological functions comprise one or more of pulse, respiration, blood pressure, body temperature, perspiration, or involuntary muscle actuation.

22. The tool of claim 20, wherein the control module controls the emission module to vary one or more aspects of the emission of the electromagnetic radiation to reflect the information related to the one or more biological functions detected by the biological function module.

23. The tool of claim 15, further comprising one or more radiation detectors that are configured to receive electromagnetic radiation and to generate one or more output signals based on one or more properties of the received electromagnetic radiation.

24. The tool of claim 15, wherein the emission module comprises one or more sources configured to emit electromagnetic radiation, and wherein the one or more sources are capable of emitting the electromagnetic radiation in a chirped fashion by modulating the amplitude of the electromagnetic radiation.

25. The tool of claim 24, wherein the one or more sources comprise one or more lasers.

26. The tool of claim 15, wherein the emission module comprises:
one or more sources configured to emit electromagnetic radiation, and
one or more gyrating elements having a reflective surface that are configured to deflect the electromagnetic radiation emitted by the one or more sources.

27. The tool of claim 15, wherein the emission module comprises:
one or more sources configured to emit electromagnetic radiation, and
a microelectromechanical system having one or more actuable reflective surfaces that are configured to deflect the electromagnetic radiation emitted by the one or more sources.

28. The tool of claim 15, further comprising one or more of a camera, a vibration sensitive device, a micro-display, a mobile telephone, a computer mouse, a temperature sensitive device, a speaker device, a hygrometer, an altimeter, or a microphone.

29. A system comprising:
a processor that causes images related to an interactive electronic game to be provided to a user;
a tool that enables a user to input control information to the processor to control one or more aspects of the interactive electronic game, wherein the user inputs control information to the processor by interacting with the tool; and
a biological function module that detects information related to one or more biological functions of the user based on the interaction of the user with the tool,
the processor altering one or more aspects of the interactive electronic game based on the information related to the one or more biological functions that is determined by the biological function module.

30. The system of claim 29, further comprising an interface surface that displays the images provided to the user, and wherein the user interacts with the tool by positioning and/or moving the tool with respect to the interface surface.

31. The system of claim 30, wherein the one or more bodily functions comprises one or both of pulse and blood pressure, and wherein the biological function module detects information related to the one or more biological functions based on the position and/or movement of the tool with respect to the interface surface as the user interacts with the tool.

32. The system of claim 29, wherein the one or more bodily functions comprise one or more of pulse, blood pressure, body temperature, or perspiration.

33. The system of claim 29, wherein the one or more bodily functions are related to one or both of a level of fatigue and a level of excitement of the user.
